(19) United States
(12) Patent Application Publication
(10) Pub. No.: US 2025/0136920 A1
(43) Pub. Date: May 1, 2025

(54) NOVEL ENHANCED PROCESSES FOR TRYPTAMINE EXTRACTION PROCESSING AND DELIVERY

(71) Applicants: Yevgeniy Galyuk, Sherman Oaks, CA (US); Maximilien Gluck, Laguna Niguel, CA (US); Kaveh Newmen, Santa Ana, CA (US)

(72) Inventors: Yevgeniy Galyuk, Sherman Oaks, CA (US); Maximilien Gluck, Laguna Niguel, CA (US); Kaveh Newmen, Santa Ana, CA (US)

(21) Appl. No.: 18/933,166

(22) Filed: Oct. 31, 2024

Related U.S. Application Data
(60) Provisional application No. 63/594,514, filed on Oct. 31, 2023.

(51) Int. Cl. C12N 1/06; A61K 31/4045; A61K 47/36; C12N 1/14

(52) U.S. Cl. CPC C12N 1/066; A61K 31/4045; A61K 47/36; C12N 1/14

(57) ABSTRACT

Tryptamine extracts and entourage effects govern mushroom and synthetically derived psychogenic and psychoactive substance delivered using dynamic thresholding dosing heuristics.

### CROSS-REFERENCE TO RELATED APPLICATIONS

The present application claims priority to U.S. Provisional Application No. 63/594,514, filed Oct. 31, 2023, the contents of which are incorporated herein by reference in their entirety.

### BACKGROUND OF THE INVENTION

In today's evolving landscape of psychedelic decriminalization and increasing interest in the therapeutic potential of substances like psilocin and psilocybin, there is a growing need to develop advanced methods for the extraction of active psychedelic compounds from the fruit and body of psychedelic mushroom strains. This demand is driven by several compelling reasons, each underscoring the importance of expanding the possibilities for medical applications through innovative delivery systems.

First and foremost, the extraction of psychoactive tryptamines such as psilocin and psilocybin from mushrooms is essential for the precise and controlled dosing necessary for medical use. Traditional methods of mushroom consumption, such as ingestion of whole mushrooms or microdosing, do not guarantee consistent or accurate dosage levels. Developing reliable extraction techniques enables the creation of pharmaceutical-grade products with known and consistent quantities of active compounds, facilitating standardized treatment regimens for various medical conditions.

Furthermore, extraction methods offer the potential for enhanced safety in the therapeutic use of psychedelics. While psychedelic mushrooms have a relatively good safety profile, adverse reactions can occur, especially in cases of overdose or allergic reactions. By isolating and purifying the active compounds, the risks associated with consuming other, potentially harmful constituents in the mushroom are significantly reduced.

The emergence of various delivery systems, such as oral pills, capsules, nasal sprays, transdermal or intravenous formulations, allows for a more targeted and controlled administration of these compounds. This, in turn, provides healthcare professionals with the tools to tailor treatment approaches to individual patient needs, optimizing efficacy and minimizing potential adverse effects.

In the broader context of medical applications, such as the treatment of depression, anxiety, PTSD, and other mental health conditions, precise dosing and controlled delivery systems are paramount. Developing robust extraction methods for mushroom native tryptamines, as well as, psilocin and psilocybin is essential to harness the full therapeutic potential of these compounds while ensuring patient safety and compliance with evolving regulations. As psychedelic decriminalization progresses, these advancements are poised to play a pivotal role in unlocking the transformative potential of these substances for the betterment of mental health treatment.

### SUMMARY OF THE INVENTION

The present invention includes a novel extraction method for the extraction of specific psychoactive compounds from mushroom strains of the psychedelic family genus.

Dried mushroom fruits and bodies, ground to a fine consistency, stored at room temperature in preparation for extraction. Moisture control is paramount to prevent the degradation of active compounds due to ambient humidity. Speed post-grinding is emphasized to minimize degradation, ensuring the preservation of the active constituents.

The present invention describes novel extraction methods and the systems thereof. The present invention employs ultrasonication of tryptamine rich biomass in a solvent mixture of methanol and H2O, although the use of other alcohols, such as ethanol or isopropyl, is not precluded.

The present invention describes key parameters such as temperature control, sonication power rate, watts, residence time, contact time.

The present invention describes additional parameters such as sonification pulse rate and solvent mixture ratios.

The present invention encompasses solubility of the active compounds in methanol and H20 with considerations for contact time and sonification power levels and sonification pulse rates.

The present invention describes post extraction steps, such as filtration of spent mushroom biomass which is devoid of all or nearly all active compounds and constituents.

The present invention describes the collection of an active rich solution or emulsion from the spent mushroom biomass.

The present invention describes active compound retrieval, achieved through methods such as rotary evaporator, ambient atmospheric evaporation, vacuum distillation, or vacuum purging of the solvent rich active solutions or emulsions.

The present invention describes an end product which is obtained in a powdered form, standardized for key tryptamines, while allowing for variations in lower-end alkaloids.

### DETAILED DESCRIPTION

The present invention uses the term “bioeffective” as defined and explained herein (likewise from those patents expressly incorporated by reference herein U.S. Pat. Nos. 10,814,248; 11,590,120; and 7,474,076) to mean having the minimum effective dose to achieve a therapeutic shift in conscious awareness via thresholding of the amounts and ratios of the subject moieties, with AI-mediated data supporting the same.

The present invention describes a novel improved method for the extraction of tryptamines from various mushroom strains rich in such. It has been discovered that pre-processing steps are crucial to the preservation of sensitive or volatile compounds such as psilocin and psilocybin.

In the present embodiment of the invention, mushroom bodies and fruits are dried as a pre-processing step, at ambient room temperature, between 60 degrees Fahrenheit and 75 degrees Fahrenheit, between 75 and 90 degrees Fahrenheit, and ideally below 100 degrees Fahrenheit. The present invention also describes an ideal humidity range of 30% atmospheric humidity to 60% atmospheric humidity, with a preferred range of 35% atmospheric humidity and 75% humidity, and an ideal target of 50% atmospheric humidity during the curing or drying process. As a secondary pre-processing step, the present embodiment of the invention requires a grinding or mulching step to break down the fruit and body of the mushroom. It has been discovered that a fine particle size permits for a greater extraction efficiency during the extraction process.

Therefore, the present embodiment of the invention requires a mechanical means of grinding the fruit and body to a fine particle size to maximize surface area which come into contact with the solvent solution. The present embodiment of the invention describes a preferred ambient temperature of 70 degrees Fahrenheit which should be maintained for the duration of the grinding process. The present embodiment of the invention describes a preferred ambient humidity between 35% and 65% for the duration of the grinding process.

The current embodiment of the invention also describes steps for storage of ground mushroom body and fruit prior to the extraction process. Preferred temperatures and humidity must be maintained during the storage and handling phase and prior to the extraction process. Similarly, speed to extraction has shown significant improvement in active compound stability. The present embodiment of the invention describes a preferred time to extraction, of 1 to 60 minutes post grinding step, preferred time of 5 to 45 minutes post grinding step, ideally between 10 and 30 minutes post grinding step.

In another embodiment of the invention an inert gas blanket is preferred to prevent oxidation when ideal temperature and humidity may not be achieved sufficiently. In this embodiment of the invention the pre-processing step of grinding is done via mechanical grinder or table top blender injected with nitrogen gas and fully purged of oxygen. In this embodiment of the invention, storage of the finely ground mushroom fruit and body biomass may be stored in inert containers purged by nitrogen gas. In this embodiment of the invention the inert nitrogen gas is preferred to be of medical quality to prevent any cross contamination of the biomass and actives therein.

In the present embodiment of the invention, dried, finely ground biomass is placed within an inert extraction container in preparation for extraction. As previously disclosed, an ideal ambient temperature is preferred prior to extraction and during the extraction. In another embodiment where ambient temperature may not be achieved, a nitrogen blanket may be applied to the extraction vessel after the biomass has been placed within.

The present invention describes an extraction vessel which is temperature regulated. This vessel may be placed in an environment box capable of achieving and maintaining a preferred or ideal temperature which is required for successful extraction. Likewise, the present invention can describe an extraction vessel which is jacketed and combined with an external circulating bath or chiller system, capable of circulating a heat transfer liquid through the vessel's jacket walls to maintain a preferred or ideal temperature range for the extraction process. In another embodiment of the present invention the extraction vessel can be sealed from atmosphere. In the same embodiment the vessel may have a removable lid with a sonication wand which protrudes through the lid and into the extraction vessel.

The present invention describes an extraction vessel which is capable of being blanketed by a nitrogen, or fully purged by vacuum to remove any oxygen from the vessel when it is filled with finely ground mushroom biomass. The present invention describes an extraction vessel suitable to withstand applied sonification via sonication probe. In another embodiment of the present invention, the extraction vessel is capable of withstanding external sonification produced by a sonification bath into which the extraction vessel is placed. In another embodiment of the present invention, a sonification bath may be used as the primary extraction vessel.

The present invention describes an embodiment where the finely ground mushroom biomass is introduced into the extraction vessel. In the present embodiment, a solvent is introduced into the extraction vessel and allowed to contact the mushroom biomass. In the present invention, a appropriate contact time has been determined to be between 3 minutes and 120 minutes, a preferred contact time between 10 to 60 minutes. The present invention teaches an important contact time factor which is determined by bursts of sonification.

The present invention describes the additional step of sonification during the extraction process. As the solvent is introduced to the extraction vessel, and contact time is allowed between the biomass and solvent, the introduction of sonification has proven to greatly increase yields whilst lowering the contact time necessary between solvent solution and finely ground mushroom biomass.

The present invention describes the solvent solution as being part Methanol and part H20. The present invention describes an acceptable ratio of methanol to be between 5% and 50%.

Therefore, a solution of 10% methanol and 90% H20 has been determined to be an effective solvent solution, but not limited to the range described. In another embodiment of the invention a solution of 20% methanol and 80% H20 can produced similar extraction efficiency when parameters of contact time and sonification are met at the ideal range.

In another embodiment of the present invention other alcohols in solution with H20 have proven to be effective in extracting the active tryptamines from mushroom biomass. Ethanol and Isopropyl alcohols may be used in solution with H20 to the same efficacy as previously described. In this embodiment of the invention ethanol and H20 have an acceptable solution ratio of 20% ethanol and 80% H20, a preferred ratio of 50% ethanol and 50% H20. In the present embodiment Isopropyl alcohol has been determined to have an acceptable ratio similar to ethanol, but may require different temperature parameters, as well as, sonification parameters.

The present invention describes the extraction process as contact time between solvent solution and finely ground mushroom biomass in a temperature-controlled environment. The present invention describes the extraction process as having an additional agitator. The present invention describes the extraction process with an introduced element of sonification. In all embodiments, agitation or sonification is preferred and sometimes ideal. In the present embodiments, agitation can be in the form of a propel mixer inserted into the solvent and biomass slurry. Similarly, an internal sonification probe can act as the preferred agitator during the extraction process. Finally, external sonification may produce an ideal agitation and extraction efficiency. In some embodiments, a nitrogen blanket is preferred to protect the actives from degradation due to oxidation. In other embodiments, proper temperature control is preferred to protect the actives from degradation during the extraction process.

In some embodiments, the solvent solution may be treated with acidic solutions to lower the overall PH of the solvent solution. Acetic acid has been show to effectively lower the PH of the solvent solution without causing unintended degradation of soluble active tryptamines within the mushroom biomass.

The post processing steps of the current invention describe filtration steps to separate the biomass from an extract-rich solution. In one embodiment, a simple Buchner funnel and filter disc may be used to separate the extract rich solution from the spent mushroom biomass after extraction. In another embodiment inline filter housings may be employed. A filter housing with a lenticular filter cartridge is ideal. A filter housing with a mesh filter cartridge may be preferred.

Similarly, the present invention describes a filtration process wherein a Nutsche filter is employed for the process of filtration. The benefits of a Nutsche filter over a Buchner funnel is the ability to deploy a nitrogen blanket and use the positive pressure to expedite the liquid-solid separation whilst minimizing oxidation.

In a preferred embodiment of the invention, the extraction vessel is connected via plumbing to an appropriate set of inline filter housings outfitted with lenticular filter cartridges capable of separating finely ground mushroom biomass from the extract-rich solution. In the same embodiment, the extraction vessel is temperature-controlled and all plumbing and filter housings are insulated. In the same embodiment all filtration liquid transfers and filtration happen in a preferred or ideal temperature range. In the same embodiment, the extraction vessel lid is retrofitted with a sonification wand that protrudes inside the vessel and can contact the mushroom biomass, which is submerged in a preferred or ideal solvent solution. In the same embodiment, the extraction vessel cavity has been purged of oxygen. In a preferred embodiment of the present description, the extraction vessel has an external sonification input to maximize the agitated area within the cavity.

The present invention describes a solvent recovery step wherein the extract-rich solution is reduced to the extract form. In one embodiment of the present invention, the extract-rich solution is reduced under vacuum in a rotary evaporator. In another embodiment, the extract-rich solution is reduced via a wiped film evaporator. In all embodiments, after reduction of the extra-rich solution to a form of super concentration, the remainder of the solvent may be purged by vacuum oven, vacuum chamber, or freeze dryer.

The present invention describes the final product obtained as an extract rich in psychedelic tryptamines such as psilocin and psilocybin.

The present invention describes the final product as being obtained via novel and effective pre-processing steps, extraction processes, liquid solid separation steps, and a final purging step to produce an active rich extract.

1. A process to establish an active rich extract, having bioeffective amounts of psychedelic tryptamines comprised of at least psilocin and psilocybin, which comprises, in combination:
providing mushroom bodies and fruits which are dried as a pre-processing step, at ambient room temperature, between 60 degrees Fahrenheit and 75 degrees Fahrenheit, between 75 and 90 degrees Fahrenheit, and ideally below 100 degrees Fahrenheit.

2. The process to establish an active rich extract, having bioeffective amounts of psychedelic tryptamines of claim 1, further comprising:
at least a secondary pre-processing step, by a grinding or mulching step to break down the fruit and body of the mushroom, because a fine particle size permits for a greater extraction efficiency during the extraction process; with prescribed storage of ground mushroom body and fruit prior to the extraction process; and, an inert gas blanket is offered to prevent oxidation when ideal temperature and humidity may not be achieved sufficiently, done with nitrogen gas and fully purged of oxygen of medical quality to prevent any cross contamination of the biomass and actives therein.

3. The process to establish an active rich extract, having bioeffective amounts of psychedelic tryptamines of claim 2, further comprising of:
an introduced element of sonification, agitation being in the form of at least one of a propel mixer inserted into the solvent and biomass slurry, an internal sonification probe and external sonification, along with proper temperature control is preferred to protect the actives from degradation during the extraction process.

4. The process to establish an active rich extract, having bioeffective amounts of psychedelic tryptamines of claim 3, further comprising:
at least a solvent solution treated with acidic solutions to lower the overall PH of the solvent solution, for example, Acetic acid has been show to effectively lower the PH of the solvent solution without causing unintended degradation of soluble active tryptamines within the mushroom biomass.

5. The process to establish an active rich extract, having bioeffective amounts of psychedelic tryptamines of claim 4, further comprising:
at least post processing filtration steps to separate the biomass from an extract-rich solution by at least one of a simple Buchner funnel and filter disc to separate the extract rich solution from the spent mushroom biomass after extraction, an in-line filter housing with a lenticular filter cartridge and a filter housing with a mesh filter, and a Nutsche filter which uses the positive pressure to expedite the liquid-solid separation whilst minimizing oxidation.

6. The process to establish an active rich extract, having bioeffective amounts of psychedelic tryptamines of claim 5, further comprising:
the extraction vessel is connected via plumbing to an appropriate set of inline filter housings outfitted with lenticular filter cartridges capable of separating finely ground mushroom biomass from the extract-rich solution.

7. The process to establish an active rich extract, having bioeffective amounts of psychedelic tryptamines of claim 1, further comprising, at least:
a solvent recovery step wherein the extract-rich solution is reduced to the extract form. In one embodiment of the present invention, the extract-rich solution is reduced under vacuum in a rotary evaporator. In another embodiment, the extract-rich solution is reduced via a wiped film evaporator. In all embodiments, after reduction of the extra-rich solution to a form of super concentration, the remainder of the solvent may be purged by vacuum oven, vacuum chamber, or freeze dryer.

8. An improved chitin-based delivery system for psychedelics, and process to extract them, which comprises, in combination:
at least an extract of tryptamine;
obtained via novel and effective pre-processing steps, extraction processes, liquid solid separation steps, and a final purging step to produce an active rich extract having an impact upon consciousness of a user or patient, tracked and recorded an integrated into an adaptable learning database mediated buy AI.

9. Products, by the process of claim 1, being inflection-point dosages of said at least an extract, whereby perceptible lability is threshold determined, in humans, by establishing at least one heuristic to the data outputting a probability that said lability event shall occur.

10. The delivery system of claim 8, wherein the at least one heuristic comprises a minute or micro-dose chemical composition causing a profound change in consciousness relating to a pre-established clinical endpoint.

11. The delivery system of claim 10, whereby the structural biochemical arrangement of the human brain exogenously reacts based upon structural biochemical aspects of the extracts.

12. Products, as defined by the process of claim 9, wherein the at least one heuristic further comprises a dynamic thresholding process as set forth in U.S. Pat. No. 7,467,076; whereby the dosemetric and user-factor bases are calculated on said basis by a special purpose computing machine.

13. In an improved dosimetrically gated composition using thresholding and heuristics to deliver resultory planned and scheduled dosages of tryptamine complexes constructed from extracts of psilocin and psilocybin and related moieties (structurally and functionally), the group consisting essentially of any effective genus and species effective from a group of Phallus duplicatus; Tidal wave, Penis envy, Phallus duplicatus, & related species, chimeras, syntheons, and the like synthetic derivatives, capable of being delivered to human in need of the same or recreationally.

14. A process to deliver the composition of claim 13, vianasal or sublingual administration, percutaneously or via patches, needles, pills, capsules, tinctures and orally or via suppository routes.

15. Compositions of claim 13, targeted at specific mental health needs of humans, and to treat addiction and depression as described herein.

16. Systems using AI-mediated data sets harvested according to claim 13.

17. Infusion sets and pumps for delivering and sensing and measuring the composition of claim 13.

18. Compositions according to claim 13, further comprised of: Tryptamine extracts from at least one of Psilocybe cubensis; bohemia—Cordyceps sp. and synthetic analogs and homologues;
in combination with delivery vehicles comprising: at least about 8 percent Chitin, and other moieties.
